(19) United States
(12) Patent Application Publication
(10) Pub. No.: US 2025/0349396 A1
(43) Pub. Date: Nov. 13, 2025

(54) DEVICE AND METHODS FOR SPECIALTY CHEMICAL DEVELOPMENT UNDER DIFFERENT TEST CONDITIONS WITH ARTIFICIAL INTELLIGENCE MODELS

(71) Applicant: ChampionX LLC, Sugar Land, TX (US)

(72) Inventors: Ming-Zhao JIN, Edmonton (CA); Peiqi QIAO, Calgary (CA); Song GAO, Calgary (CA)

(73) Assignee: ChampionX LLC, Sugar Land, TX (US)

(21) Appl. No.: 19/196,051

(22) Filed: May 1, 2025

Related U.S. Application Data
(60) Provisional application No. 63/643,870, filed on May 7, 2024.

(51) Int. Cl. G16C 20/30; G16C 20/50

(52) U.S. Cl. CPC G16C 20/30; G16C 20/50

(57) ABSTRACT

Technologies for specialty chemical development and testing include devices and methods for normalizing historical specialty chemical test results and training a chemical composition predictor to predict chemical components of a formulation given a test condition and a normalized performance indicator based on the normalized test results. The specialty chemical may be a corrosion indicator, and the normalized performance indicator may be corrosion rate. The devices and methods may predict a predicted composition with the trained chemical composition predictor. the devices and methods may filter the normalized test results based on the predicted composition and train a formulation optimization predictor to predict a normalized performance indicator based on the filtered test results. The devices and methods may generate multiple candidate chemical formulations based on the predicted composition and predict a normalized performance indicator for each candidate chemical formulation with the trained formulation optimization predictor.

### BACKGROUND

Several types of specialty chemicals, such as demulsifiers, corrosion inhibitors, scale inhibitors, paraffin inhibitors, and defoamers, are used during oil and/or gas production. Due to complicated formulation and application scenarios, selection and development of the specialty chemicals is typically an empirical process.

### SUMMARY

The present disclosure provides a computing device for specialty chemical formulation development that includes a data preparation module and a chemistry composition prediction module. The data preparation module is to normalize a plurality of historical specialty chemical test results to generate normalized test results, wherein each normalized test result is indicative of a test condition and a normalized performance indicator. The chemistry composition prediction module is to train a chemical composition predictor to predict a plurality of chemical components of a formulation given a test condition and a normalized performance indicator based on the normalized test results. The chemistry composition module is further to predict a predicted composition given a specified test condition and a specified normalized performance indicator with the chemical composition predictor in response to training of the chemical composition predictor, wherein the predicted composition is indicative of a plurality of chemical components.

In an embodiment, the test condition comprises pressure, temperature, pH, or bicarbonate (HCO₃) concentration. In an embodiment, the predicted composition is further indicative of a probability of passing the specified normalized performance indicator at the specified test condition for each chemical component of the plurality of chemical components. In an embodiment, the historical specialty chemical test results comprise corrosion inhibitor test results, and wherein the normalized performance indicator is indicative of a measured corrosion rate scaled between a predetermined minimum value and a predetermined maximum value.

In an embodiment, to normalize the plurality of historical chemical test results comprises to perform absolute normalization of the historical test results based on a predetermined threshold value. In an embodiment, to normalize the plurality of historical chemical test results comprises to perform conditional normalization of the historical test results for a predetermined test condition. In an embodiment, to normalize the plurality of historical chemical test results comprises to average the absolute normalization and the conditional normalization.

In an embodiment, to train the chemical composition predictor comprises to categorize each chemical component of each formulation of the normalized test results into a component category based on relative proportion of each chemical component. In an embodiment, to categorize each chemical component comprises to categorize each chemical component as a major component, a medium component, or a minor component based on a component percentage in each formulation, wherein each component category has a substantially equal number of components. In an embodiment, to train the chemical composition predictor comprises to train a plurality of machine learning models, wherein each machine learning model of the plurality of machine learning models is trained to predict a corresponding component category. In an embodiment, to train the chemical composition predictor comprises to train a first machine learning model to predict a corresponding first component category based on the normalized test results and to train a second machine learning model to predict a corresponding second component category based on the normalized test results and an output from the first machine learning model.

In an embodiment, the computing device further includes a formula optimization module that is to filter the normalized test results based on the predicted composition to generate filtered test results, wherein the filtered test results are based on historical specialty chemical tests that involve the plurality of chemical components of the predicted composition. The formula optimization module is further to train a formulation optimization predictor to predict a normalized performance indicator given a test condition and a formulation based on the filtered test results, wherein the formulation is indicative of a percentage composition for each chemical component. The formula optimization module is further to generate a plurality of candidate chemical formulations based on the predicted composition, wherein each candidate chemical formulation is indicative of a percentage composition for each chemical component of the predicted composition; and to predict a predicted normalized performance indicator for each of the candidate chemical formulations given a requested test condition and a respective candidate chemical formulation with the formulation optimization predictor in response to training of the formulation optimization predictor.

In an embodiment, to generate the plurality of candidate chemical formulations comprises to generate a one-hot encoding of a representation of the plurality of candidate chemical formulations. In an embodiment, the formula optimization module is further to identify a top performing candidate chemical formulation based on the predicted normalized performance indicator. In an embodiment, to identify the top performing candidate chemical formulation comprises to sort the plurality of candidate chemical formulations by predicted normalized performance indicator. In an embodiment, to identify the top performing candidate comprises to cluster the plurality of candidate chemical formulations and to select the top performing candidate from a cluster with high normalized performance indicators. In an embodiment, the formula optimization module is further to receive additional test results associated with the top performing candidate chemical formulation; and re-train the formulation optimization predictor based on the additional test results.

According to another aspect of the disclosure, a method for specialty chemical formulation development includes normalizing, by a computing device, a plurality of historical specialty chemical test results to generate normalized test results, wherein each normalized test result is indicative of a test condition and a normalized performance indicator; training, by the computing device, a chemical composition predictor to predict a plurality of chemical components of a formulation given a test condition and a normalized performance indicator based on the normalized test results; and predicting, by the computing device, a predicted composition given a specified test condition and a specified normalized performance indicator with the chemical composition predictor in response to training the chemical composition predictor, wherein the predicted composition is indicative of a plurality of chemical components.

In an embodiment, the test condition comprises pressure, temperature, pH, or bicarbonate (HCO₃) concentration. In an embodiment, the predicted composition is further indicative of a probability of passing the specified normalized performance indicator at the specified test condition for each chemical component of the plurality of chemical components. In an embodiment, the historical specialty chemical test results comprise corrosion inhibitor test results, and wherein the normalized performance indicator is indicative of a measured corrosion rate scaled between a predetermined minimum value and a predetermined maximum value.

In an embodiment, normalizing the plurality of historical chemical test results comprises performing absolute normalization of the historical test results based on a predetermined threshold value. In an embodiment, normalizing the plurality of historical chemical test results comprises performing conditional normalization of the historical test results for a predetermined test condition. In an embodiment, normalizing the plurality of historical chemical test results comprises averaging the absolute normalization and the conditional normalization.

In an embodiment, training the chemical composition predictor comprises categorizing each chemical component of each formulation of the normalized test results into a component category based on relative proportion of each chemical component. In an embodiment, categorizing each chemical component comprises categorizing each chemical component as a major component, a medium component, or a minor component, wherein each component category has a substantially equal number of components. In an embodiment, training the chemical composition predictor comprises training a plurality of machine learning models, wherein each machine learning model of the plurality of machine learning models is trained to predict a corresponding component category. In an embodiment, training the chemical composition predictor comprises training a first machine learning model to predict a corresponding first component category based on the normalized test results and training a second machine learning model to predict a corresponding second component category based on the normalized test results and an output from the first machine learning model.

In an embodiment, the method further comprises filtering, by the computing device, the normalized test results based on the predicted composition to generate filtered test results from the entire training data set including all chemistry, wherein the filtered test results are based on historical specialty chemical tests that involve the plurality of chemical components of the predicted composition; training, by the computing device, a formulation optimization predictor to predict a normalized performance indicator given a test condition and a formulation based on the filtered test results, wherein the formulation is indicative of a percentage composition for each chemical component; generating, by the computing device, a plurality of candidate chemical formulations based on the predicted composition, wherein each candidate chemical formulation is indicative of a percentage composition for each chemical component of the predicted composition; and predicting, by the computing device, a predicted normalized performance indicator for each of the candidate chemical formulations given a requested test condition and a respective candidate chemical formulation with the formulation optimization predictor in response to training the formulation optimization predictor.

In an embodiment, generating the plurality of candidate chemical formulations comprises one-hot encoding a representation of the plurality of candidate chemical formulations. In an embodiment, the method further comprises identifying, by the computing device, a top performing candidate chemical formulation based on the predicted normalized performance indicator. In an embodiment, identifying the top performing candidate chemical formulation comprises sorting the plurality of candidate chemical formulations by predicted normalized performance indicator. In an embodiment, identifying the top performing candidate comprises clustering the plurality of candidate chemical formulations and selecting the top performing candidate from a cluster with high normalized performance indicators. In an embodiment, the method further comprises receiving, by the computing device, additional test results associated with the top performing candidate chemical formulation; and re-training, by the computing device, the formulation optimization predictor based on the additional test results.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of a system for specialty chemical formulation development;

FIG. 2 is a simplified block diagram of an environment that may be established by a computing device of the system of FIG. 1;

FIGS. 3 and 4 are exemplary flow diagrams of at least one embodiment of a method for specialty chemical formulation development that may be executed by the computing device of FIGS. 1 and 2;

FIG. 5 is a schematic diagram illustrating predicted chemical compositions that may be generated by the system of FIGS. 1-4; and

FIG. 6 is a schematic diagram illustrating predicted chemical compositions that may be generated by the system of FIGS. 1-4.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors or processing units (e.g., GPUs, or tensor processing units (TPUs)). A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

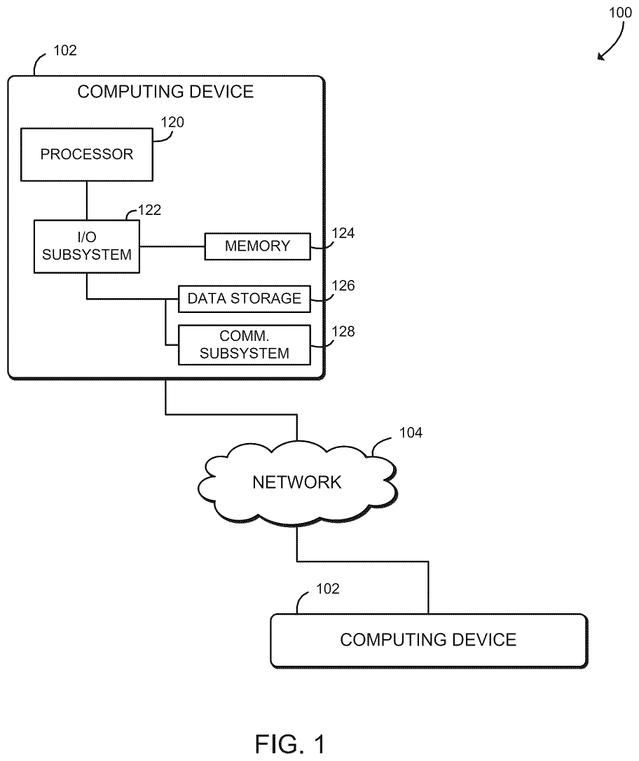

Referring now to FIG. 1, an illustrative system 100 includes a computing device 102 that may be in communication with multiple additional computing devices 102 over a network 104. In use, as described further below, the computing device 102 normalizes a database of historical test results and trains a chemical composition predictor to predict chemical components of a formulation given a test condition and normalized performance indicator. As described further below, such databases may include data lakes or databases such as SQL, NoSQL, or the like. A tester may provide a test condition for a test of a specialty chemical to a computing device 102 and a normalized performance indicator, for example through a website or other client-server interface, or alternatively directly with a user interface of the computing device 102. The computing device 102 predicts a chemical composition with the trained chemical composition predictor based on the specified test condition and normalized performance indicator. Additionally, the computing device 102 may filter the normalized test results based on the predicted chemical composition and further train a formulation optimization predictor to predict a normalized performance indictor based on the filtered test results. The formulation optimization predictor may be used to predict a normalized performance indicator for each of multiple candidate chemical formulations. Thus, the system 100 may provide a platform with machine-learning technology to enable improved development and testing of specialty chemicals, such as oil field specialty chemicals. In particular, the system 100 enables a shortened development/selection process and may lead to increase performance of resulting specialty chemicals.

The computing device 102 may be embodied as any type of device capable of performing the functions described herein. For example, a computing device 102 may be embodied as, without limitation, a server, a rack-mounted server, a blade server, a workstation, a network appliance, a web appliance, a desktop computer, a laptop computer, a tablet computer, a smartphone, a consumer electronic device, a distributed computing system, a multiprocessor system, and/or any other computing device capable of performing the functions described herein. Additionally, in some embodiments, the computing device 102 may be embodied as a “virtual server” formed from multiple computing devices distributed across the network 104 and operating in a public or private cloud. Accordingly, although each computing device 102 is illustrated in FIG. 1 as embodied as a single computing device, it should be appreciated that each computing device 102 may be embodied as multiple devices cooperating together to facilitate the functionality described below. As shown in FIG. 1, the illustrative computing device 102 includes a processor 120, an I/O subsystem 122, memory 124, a data storage device 126, and a communication subsystem 128. Of course, the computing device 102 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 124, or portions thereof, may be incorporated in the processor 120 in some embodiments.

The processor 120 may be embodied as any type of processor or compute engine capable of performing the functions described herein. For example, the processor may be embodied as a single or multi-core processor(s), digital signal processor, microcontroller, or other processor or processing/controlling circuit. Similarly, the memory 124 may be embodied as any type of volatile or non-volatile memory or data storage capable of performing the functions described herein. In operation, the memory 124 may store various data and software used during operation of the computing device 102 such as operating systems, applications, programs, libraries, and drivers. The memory 124 is communicatively coupled to the processor 120 via the I/O subsystem 122, which may be embodied as circuitry and/or components to facilitate input/output operations with the processor 120, the memory 124, and other components of the computing device 102. For example, the I/O subsystem 122 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 122 may form a portion of a system-on-a-chip (SoC) and be incorporated, along with the processor 120, the memory 124, and other components of the computing device 102, on a single integrated circuit chip.

The data storage device 126 may be embodied as any type of device or devices configured for short-term or long-term storage of data such as, for example, memory devices and circuits, memory cards, hard disk drives, solid-state drives, or other data storage devices. The communication subsystem 128 of the computing device 102 may be embodied as any communication circuit, device, or collection thereof, capable of enabling communications between the computing device 102 and other remote devices. The communication subsystem 128 may be configured to use any one or more communication technology (e.g., wireless or wired communications) and associated protocols (e.g., Ethernet, InfiniBand® Bluetooth®, Wi-Fi®, WiMAX, 3G LTE, 5G, etc.) to effect such communication.

As discussed in more detail below, the computing devices 102 may be configured to transmit and receive data with each other and/or other devices of the system 100 over the network 104. The network 104 may be embodied as any number of various wired and/or wireless networks. For example, the network 104 may be embodied as, or otherwise include, a wired or wireless local area network (LAN), a wired or wireless wide area network (WAN), a cellular network, and/or a publicly-accessible, global network such as the Internet. As such, the network 104 may include any number of additional devices, such as additional computers, routers, stations, and switches, to facilitate communications among the devices of the system 100.

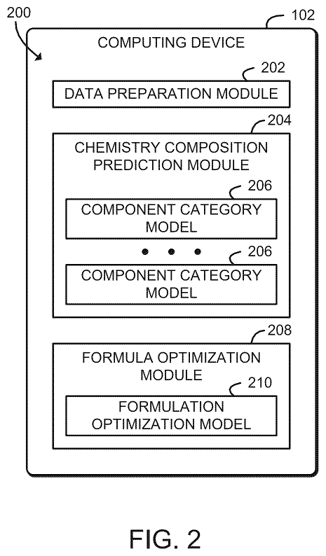

Referring now to FIG. 2, in the illustrative embodiment, the computing device 102 establishes an environment 200 during operation. The illustrative environment 200 includes a data preparation module 202, a chemistry composition prediction module 204, and a formula optimization module 208. The various components of the environment 200 may be embodied as hardware, firmware, software, or a combination thereof. As such, in some embodiments, one or more of the components of the environment 200 may be embodied as circuitry or a collection of electrical devices (e.g., data preparation circuitry 202, chemistry composition prediction circuitry 204, and/or formula optimization circuitry 208). It should be appreciated that, in such embodiments, one or more of those components may form a portion of the processor 120, the I/O subsystem 122, and/or other components of the computing device 102.

The data preparation module 202 is configured to normalize historical specialty chemical test results to generate normalized test results. Each normalized test result is indicative of a test condition and a normalized performance indicator. For example, in an embodiment, the historical specialty chemical test results are corrosion inhibitor test results, and the normalized performance indicator is indicative of a measured corrosion rate scaled between a predetermined minimum value and a predetermined maximum value. In some embodiments, normalizing the historical chemical test results may include performing absolute normalization based on a predetermined threshold value, performing conditional normalization, and/or averaging the absolute normalization and the conditional normalization.

The chemistry composition prediction module 204 is configured to train a chemical composition predictor to predict chemical components of a formulation given a test condition and a normalized performance indicator, based on the normalized test results. The test condition may include pressure, temperature, pH, bicarbonate (HCO₃) concentration, or other physical or chemical parameters. The chemical composition predictor may include one or more component category models 206 as described further below. In some embodiments, training the chemical composition predictor may include categorizing each chemical component of each formulation of the normalized test results into a component category based on relative proportion of each chemical component, such as a major component, a medium component, or a minor component, where each component category has a substantially equal number of components. In some embodiments, training the chemical composition predictor may include training multiple machine learning models 206, where each machine learning model 206 is trained to predict a corresponding component category. In some embodiments, one of the machine learning models 206 (e.g., a major component model) may be trained to predict a corresponding component category based on the normalized test results, and another machine learning model 206 (e.g., a medium component model) may be trained to predict a corresponding component category based on the normalized test results and an output from the other machine learning model.

The chemistry composition prediction module 204 is further configured to predict a predicted composition given a specified test condition and a specified normalized performance indicator using the trained chemical composition predictor. The predicted composition is indicative of the chemical components of a formulation. In some embodiments, the predicted composition may be further indicative of a probability for each chemical component of passing the specified normalized performance indicator at the specified test condition.

The formula optimization module 208 is configured to filter the normalized test results based on the predicted composition to generate filtered test results. The filtered test results are based on historical specialty chemical tests that involve the chemical components of the predicted composition. The formula optimization module 208 is further configured to train a formulation optimization predictor (such as a formulation optimization model 210) to predict a normalized performance indicator given a test condition and a formulation based on the filtered test results. The formulation is indicative of a percentage composition for each chemical component. The formula optimization module 208 is further configured to generate multiple candidate chemical formulations based on the predicted composition. Each candidate chemical formulation is indicative of a percentage composition for each chemical component of the predicted composition. In some embodiments, generating the candidate chemical formulations may include generating a one-hot encoding of a representation of the candidate chemical formulations. The formula optimization module 208 is further configured to predict a predicted normalized performance indicator for each of the candidate chemical formulations given a requested test condition and a respective candidate chemical formulation using the trained formulation optimization predictor. The formula optimization module 208 may be further configured to identify a top performing candidate chemical formulation based on the predicted normalized performance indicator, for example by sorting the candidate chemical formulations by predicted normalized performance indicator or by clustering the candidate chemical formulations and selecting the top performing candidate from a cluster with high normalized performance indicators. The formula optimization module 208 may be further configured to receive additional test results associated with the top performing candidate chemical formulation and to re-train the formulation optimization predictor based on the additional test results.

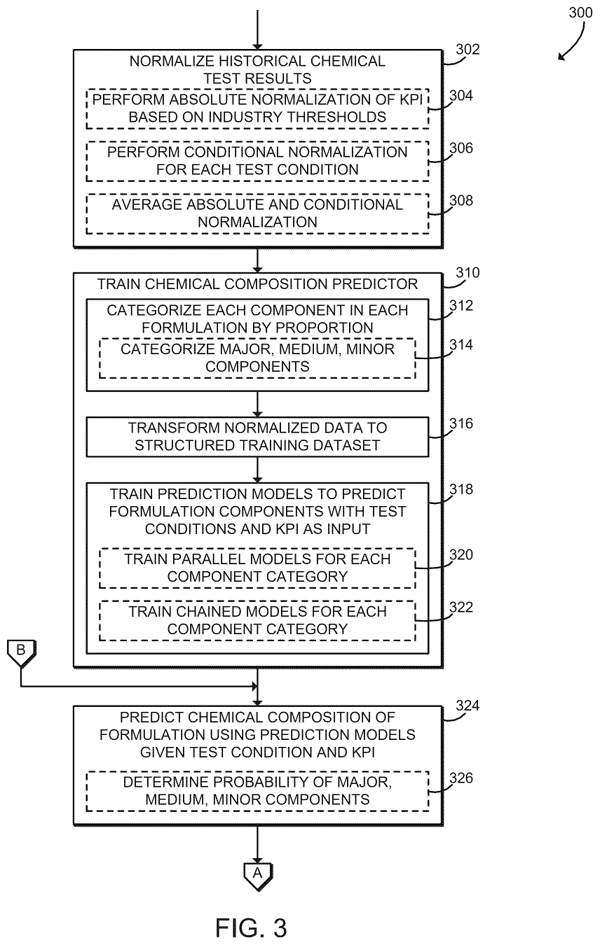

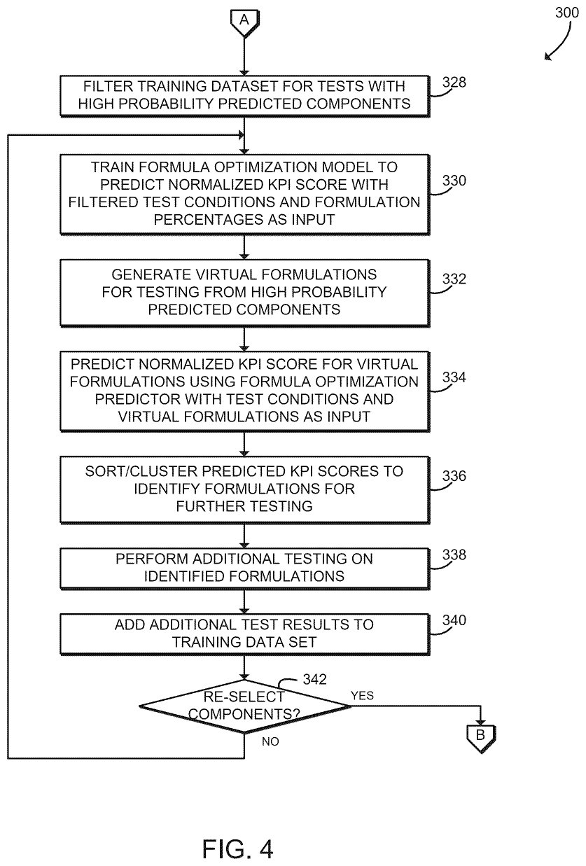

Referring now to FIGS. 3 and 4, in use, the computing device 102 may execute a method 300 for specialty chemical formulation development. It should be appreciated that, in some embodiments, the operations of the method 300 may be performed by one or more components of the environment 200 of the computing device 102 as shown in FIG. 2. The method 300 begins with block 302, in which the computing device 102 normalizes a multiple historical chemical test results. The test results are based on experimental test results for a test of an oil field specialty chemical, such as a demulsifier, a scale inhibitor, a paraffin inhibitor, a dispersant, a corrosion inhibitor, a defoamer, and/or other specialty chemical. The historical test results may be stored in a relational database, an object database, a data lake, a database such as SQL, NoSQL, or other data store accessible by the computing device 102. Each test result may be associated with a historical test of a specialty chemical and thus may include information related to the test parameters of the historical test, the historical formulation that was tested, historical test result performance data, including values for key performance indicators, or other information related to the historical test. Accordingly, each test result is indicative of one or more key performance indicators (KPIs) associated with the specialty chemical. For example, for historical test results associated with a corrosion inhibitor, the test results may be indicative of corrosion rage (e.g., in milli-inches per year (mpy)). The specialty chemical may be represented by a formulation of chemical components, for example indicating the percentage of each component included in the specialty chemical. The test results are also associated with one or more test conditions or other parameters associated with the test, such as one or more physical or chemical parameters such as pressure, temperature, acidity (pH), bicarbonate (HCO₃) concentration, geometrical location, or other test parameters.

Normalizing the test results may include scaling one or more KPIs to a predetermined numeric scale (e.g., from 0 to 100 or other scale). Normalization effectively translates raw test data KPIs into a standardized score, for example from 0 to 100, with 0 representing the poorest performance and 100 representing optimal performance. The normalized, uniform scoring metric may enhance sequent analysis, for example, by ensuring that a positive shift in score indicates an improvement in performance, or by streamlining the weighting process for scenarios with multiple KPIs.

In some embodiments, in block 304, the computing device 102 may perform absolute normalization of one or more key performance indicators (KPI) based on industry thresholds. For example, test results may be compared to an industry standard performance threshold, and test results may be scaled based on that comparison. Continuing that example, for corrosion inhibitors, a common industry standard threshold corrosion rate is 2.0 milli-inches per year (mpy). A statistical analysis of the historical test results determined that the median of lowest corrosion rates was 0.9 mpy, and the median of highest corrosion rates was 8.8 mpy. Continuing that example, the corrosion rates of the test results may be mapped such that corrosion rates≤0.9 mpy are mapped to a score of 100, corrosion rates of 2.0 are mapped to a score of 60, and corrosion rates≥8.8 mpy are mapped to a score of 0. Accordingly, this absolute normalization maps test results onto a scale ranging from 0 (indicating poorest performance) to 100 (indicating optimal performance). Of course, other absolute normalization schemes are possible in other embodiments.

In some embodiments, in block 306, the computing device 102 may perform conditional normalization for each test condition. In such embodiments, normalization may be constrained within the parameters of each distinct test condition. For example, the lowest-performing chemical for a certain test condition may be assigned a score of 0, and the highest-performing chemical for that test condition may be assigned a score of 100. Test conditions may significantly influence KPIs of each specialty chemical formulation. For example, in a certain test condition for testing corrosion inhibitors, the minimum observed corrosion rate was 5.1 mpy, which may be assigned the highest score of 100, even though the observed corrosion rate is markedly above the industry standard threshold of 2.0 mpy. Conditional normalization may thus allow trained prediction models to predict the most effective chemistry for specified test conditions, even when the most effective chemical or product does not meet the industry standard threshold in those test conditions.

In some embodiments, in block 308, the computing device 102 may average the absolute normalization and the conditional normalization that were determined as described above. For example, the computing device 102 may generate an equal-weighted average of the two normalization methods or other weighted average.

In block 310, the computing device 102 trains a chemical composition predictor using the normalized historical test results. The chemical composition predictor may be embodied as one or more machine learning models, such as a regressor, a classifier, an artificial neural network, a support vector machine, linear regression, logistic regression, or any other supervised machine learning prediction model. As described further below, those machine learning models may be illustratively used in a parallel configuration or in a chained configuration. In block 312, the computing device 102 categorizes each component in each chemical formulation in the normalized test results by proportion (e.g., percentage of each component in formulation). In some embodiments, in block 314 the computing device 102 may categorize the components into three categories: major components, medium components, and minor components. The components may be categorized using the percentage distribution across the entire component population from the historical test data such that equal (or similar) numbers of components are included in each category. This balanced distribution of components may enhance the quality of machine learning training. For example, in an illustrative set of test data, major components included those components having a component percentage above 21.4%, medium components included component percentages from 7.3% to 21.4%, and minor components included component percentages below 7.3%. Other categorization criteria may be used, for example categorizing major components as those with component percentage above 20%, medium components as those in the range from 5-20%, and minor components as those below 5%. As another example, components having a relatively high percentage (e.g., 40-100%) may be categorized as a major component, components having a medium percentage (e.g., 10-40%) as a medium component, and components having a lower percentage (e.g., 0.5-10%) as a minor component. Of course, other embodiments may use a different number of categories (e.g., less than three or more than three categories).

In block 316, the computing device 102 transforms the normalized data into a structured training dataset. The structured training dataset is suitable for training the one or more machine learning models of the chemical composition predictor. For example, the structured training dataset may identify model inputs for a test result, including test conditions and normalized score (i.e., normalized KPI). The structured training dataset may also identify one or more model targets for the test, including a chemical component from the specialty chemical formulation for each component category (e.g., a major component, a medium component, and a minor component). As another example, transforming to structured training dataset may allow for the selection of various data formats for formulation integration, catering to diverse model requirements. For example, while the corrosion inhibitor example primarily utilizes active component formulations, in other embodiments, solvent formulations may be used when training material compatibility models. The specialty chemical formulation may also be represented in at least two distinct formats within the machine learning data structure. For example, one format may specify “50% resin and 50% sorbitol,” encompassing a less detailed chemical categorization. Another format may delineate the formulation as “30% resin A+20% resin B+25% sorbitol A+25% sorbitol B,” where “resin A” and “resin B” are subtypes within the resin category, and “sorbitol A” and “sorbitol B” represent different components under the sorbitol category. Accordingly, the computing device 102 allows for a high degree of customization in data representation, ensuring that the model can be tailored to specific requirements and applications.

In block 318, the computing device 102 trains one or more machine learning prediction models to predict formulation components with test conditions and a KPI (normalized score) or KPI range as inputs. Each predicted formulation component represents a particular chemical component that may be included in a specialty chemical formulation in a specified category (e.g., a major component, a medium component, or a minor component). Additionally, in some embodiments the machine learning models may be trained to predict a percentage probability associated with each predicted formulation component. The computing device 102 may use any appropriate machine learning algorithm to train the machine learning models, such as an ensemble learning method (e.g., AdaBoost), or a gradient boosting algorithm (e.g., CatBoost).

In some embodiments, in block 320, the computing device 102 may train multiple, parallel machine learning models. In the parallel models configuration, each machine learning model is independently trained for a particular component category. For example, in the illustrative embodiment, the computing device 102 trains three machine learning models in parallel: one model each for the major component, medium component, and minor component categories. In some embodiments, in block 322 the computing device 102 may train multiple, chained machine learning models. In the chained models configuration, output from a trained model for a component category is used to train models for subsequent categories. For example, in the illustrative embodiment, the computing device 102 may train a single shared model for major components (similar to the parallel model configuration). Based on the test condition and the specified range of normalized scores, predicted information from the major component model is incorporated into a new training dataset, which is used to train the model for predicting the medium component model. Similarly, predicted output from both the major component model and the medium component model is used to train the minor component model.

In block 324, after training the chemical composition predictor, the computing device 102 predicts chemical composition of a specialty chemical formulation using the trained chemical composition predictor given a test condition and a specified KPI or KPI range. For example, the specified KPI range may identify normalized scores between 60 to 100, which may be associated with performance above an industry standard threshold or otherwise acceptable performance. As another example, the KPI or KPI range may identify particular numerical KPI values (e.g., between 0.0 to 2.0 mpy for corrosion inhibitors) or may provide a categorical KPI (e.g., a “pass” or other acceptable value). The chemical composition predictor may perform prediction using machine learning models in a parallel configuration or a chained configuration as described above. In some embodiments, in block 326 the computing device 102 may determine a probability associated with each component of the formulation (e.g., each of the major component, medium component, and minor component). For example, in an ensemble learning method like AdaBoost, the probability of a predicted component may be estimated by aggregating the weighted votes attributed to that component and subsequently normalizing these sums. This approach essentially leverages the collective decision-making of an ensemble of weak learners to arrive at a probability estimate. As another example, algorithms based on gradient boosting, such as CatBoost, may compute the probabilities of predicted components by transforming the cumulative sum of gradients or corrections contributed by each tree in the ensemble. This transformation may be accomplished through a logistic function, effectively converting the sum of iterative improvements made by the ensemble into a probability measure.

In block 328, shown in FIG. 4, the computing device 102 filters the training dataset to include tests with high-probability predicted components. For example, the computing device 102 may identify chemical components in the predicted chemical composition having an associated probability greater than a threshold or otherwise having a high probability. The computing device 102 may identify chemical components from all categories (e.g., major components, medium components, and minor components). After identifying the high probability chemical components, the computing device 102 may filter the normalized test results to include only test results that use any one or more of the high probability chemical components.

In block 330, the computing device 102 trains a formulation optimization predictor to predict normalized KPI score with the filtered, normalized test conditions and associated formulation percentages as inputs. The formulation optimization predictor may be embodied as one or more machine learning prediction models, such as a regressor, a classifier, an artificial neural network, a support vector machine, linear regression, logistic regression, or any other supervised machine learning prediction model. The computing device 102 may, for example, extract test conditions and formulation percentages for the filtered test results as input features. In some embodiments, the formulation percentages may be encoded using a one-hot encoding or other feature encoding technique. The model training target is the normalized KPI score, such as the normalized corrosion rate score for corrosion inhibitors as described above. The computing device 102 may use any appropriate machine learning method to train the machine learning prediction model.

In block 332, the computing device 102 generates multiple virtual chemical formulations or formulation candidates based on the high-probability predicted components of the predicted chemical composition. Each virtual formulation identifies one or more constituent chemicals or other components of the formulation, and a corresponding proportion of that constituent chemical. As an illustrative example, the computing device 102 may generate all potential virtual formulations that include all possible combinations of the identified high-probability chemical components, over a range of percentages with a particular percentage accuracy. Continuing that example, in an embodiment, the high-probability predicted components may include Component A, Component B, and Component C, which are major, medium, and minor components, respectively. In an example, the computing device 102 may generate multiple virtual formulations as shown below in Table 1. Of course, as the number of high-probability chemical components increases, the number of virtual formulations may also increase. The computing device 102 may feasibly generate all possible combinations of high-probability chemical components due to filtering the test results as described above.

TABLE 1
Illustrative virtual formulations.

| Virtual Formulation | Component A | Component B | Component C |
|---|---|---|---|
| VF01 | 95% | 5% | 0% |
| VF02 | 90% | 5% | 5% |
| VF03 | 90% | 10% | 0% |
| VF04 | 85% | 10% | 5% |
| VF05 | 85% | 15% | 0% |
| VF06 | 80% | 15% | 5% |
| VF07 | 80% | 20% | 0% |

. . .

In block 334, the computing device 102 predicts a normalized KPI score for each of the virtual formulations using the trained formulation optimization predictor with a specified set of test conditions and the virtual formulation as inputs. For example, continuing the corrosion inhibitor example, the computing device 102 may determine a normalized corrosion rate score for each virtual formulation for a specified test condition.

In block 336, the computing device 102 sorts and/or clusters predicted KPI scores to identify virtual formulation candidates for further testing. For example, the computing device 102 may sort the virtual formulations by predicted score to identify those virtual formulations with the highest predicted performance. As another example, the computing device 102 may cluster the virtual formulations to identify similar formulations, for example using an unsupervised machine learning algorithm. The virtual formulations may be clustered based on one or more features of the formulation, such as a chemical type, a molecular weight, a chemical code, a numeric feature, or other feature. The computing device 102 may select a centroid formulation from a cluster of high-performing virtual formulations. The identified virtual formulations may be used for further recommendation, testing, or trial purposes.

For example, in some embodiments, in block 338 additional tests may be performed on the identified virtual formulations. Continuing that example, a tester or other user may perform one or more additional tests on a chemical based on an identified virtual formulation. In block 340, the computing device 102 may receive additional test results associated with the tested virtual formulation, and may add those additional test results to the historical test results. The test results may be received, for example, from the tester or other user via a web interface of the computing device 102, or other interface of the computing device 102.

In block 342, the computing device 102 determines whether to re-select components for formula optimization. For example, the computing device 102 may re-select components if performance of testing based on the current composition remains poor after multiple iterations of formula optimization or based on other performance criteria. If the computing device 102 determines to re-select components, the method 300 loops back to block 324, shown in FIG. 3, in which the computing device 102 may re-select a chemical composition, filter the normalized test results (including additional test results), and retrain the formulation optimization predictor. Referring again to block 342, if the computing device 102 determines not to re-select components, the method 300 loops back to block 330, in which the computing device 102 may retrain the formulation optimization predictor based on the updated test results (without changing component selection or the filtered dataset). This retraining based on additional test results is feasible due to the filtering performed by the computing device 102 to reduce the training dataset to include results relative to high-probability predicted components. Accordingly, the method 300 may allow for continued development of specialty chemicals.

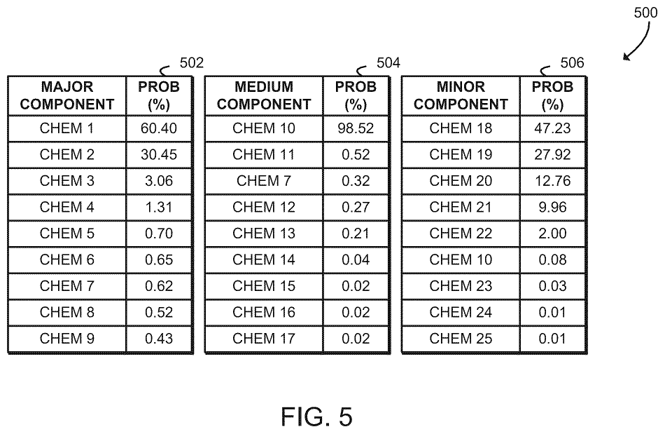

Referring now to FIG. 5, diagram 500 illustrates one potential embodiment of predicted chemical compositions that may be generated by the computing device 102 with the chemical composition predictor as described above. The diagram 500 illustrates results generated by the chemical composition predictor with component category models arranged in the parallel configuration. Accordingly, the illustrative results include major component results 502 generated by a major component model, medium component results 504 generated by a medium component model, and minor component results 506 generated by a minor component model. Each of the results 502, 504, 506 may be generated independently (e.g., in parallel) based on the normalized test results. As shown, each predicted result identifies a chemical component (e.g., Chem 1, Chem 2, etc.) and an associated probability. As shown, chemical components may be included in more than one category (e.g., Chem 7 included as a major component or a medium component; or Chem 10 included as a medium component or a minor component). As described above, the predicted chemical compositions of FIG. 5 may be used to filter the normalized test results for training the formulation optimization predictor. For example, high-probability chemical components may be identified based on the results 502, 504, 506 (e.g., Chem 1, Chem 2, Chem 10, Chem 18, and Chem 19), and the normalized test results may be filtered for those high-probability chemical components.

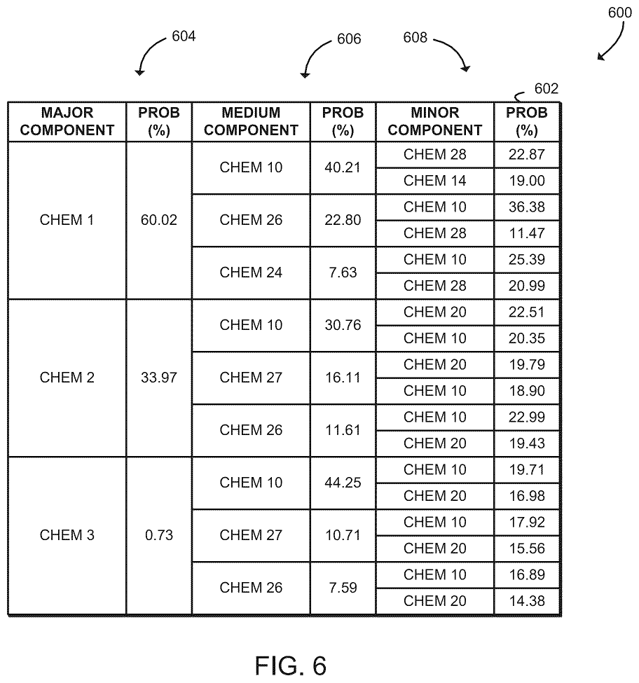

Referring now to FIG. 6, diagram 600 illustrates one potential embodiment of predicted chemical compositions that may be generated by the computing device 102 with the chemical composition predictor as described above. The diagram 600 illustrates results 602 generated by the chemical composition predictor with component category models arranged in the chained configuration. Accordingly, the illustrative results include major component results 604 generated by a major component model. The major component results 604 are used as an input to the medium component model to generate medium component results 606. Similarly, the medium component results 606 are used as an input to a minor component model to generate minor component results 608. As shown, the predicted results 604 identify major components Chem 1, Chem 2, Chem 3 with associated probabilities. The predicted results 606 identify medium components in combination with each of the major components (e.g., Chem 10, Chem 26, and Chem 24 with Chem 1; Chem 10, Chem 27, and Chem 26 with Chem 2; and so on) with associated probabilities. Similarly, the predicted results 608 identify minor components in combination with each of the major components and medium components (e.g., Chem 28 with Chem 1 and Chem 10; Chem 14 with Chem 1 and Chem 10; Chem 10 with Chem 1 and Chem 26; Chem 28 with Chem 1 and Chem 26; and so on). As described above, the predicted chemical compositions of FIG. 6 may be used to filter the normalized test results for training the formulation optimization predictor. For example, high-probability chemical components may be identified based on the results 602, and the normalized test results may be filtered for those high-probability chemical components. As shown, in the illustrative embodiment the chained configuration and the parallel configuration shown in FIG. 5 both indicate that Chem 1 and Chem 2 are high-probability major components. However, the probabilities for medium components and minor components shown in FIG. 6 vary compared to the results of FIG. 5 depending on the selected major component. This variation is attributable to the chain model's training process, which incorporates test conditions and the predicted major component, and thus may capture a synergistic relationship between major and medium components.

What is claimed is:

1. A computing device for specialty chemical formulation development, the computing device comprising:
a data preparation module to normalize a plurality of historical specialty chemical test results to generate normalized test results, wherein each normalized test result is indicative of a test condition and a normalized performance indicator; and
a chemistry composition prediction module to (i) train a chemical composition predictor to predict a plurality of chemical components of a formulation given a test condition and a normalized performance indicator based on the normalized test results, and (ii) predict a predicted composition given a specified test condition and a specified normalized performance indicator with the chemical composition predictor in response to training of the chemical composition predictor, wherein the predicted composition is indicative of a plurality of chemical components.

2. The computing device of claim 1, wherein the test condition comprises pressure, temperature, pH, or bicarbonate (HCO₃) concentration.

3. The computing device of claim 1, wherein the predicted composition is further indicative of a probability of passing the specified normalized performance indicator at the specified test condition for each chemical component of the plurality of chemical components.

4. The computing device of claim 1, wherein the historical specialty chemical test results comprise corrosion inhibitor test results, and wherein the normalized performance indicator is indicative of a measured corrosion rate scaled between a predetermined minimum value and a predetermined maximum value.

5. The computing device of claim 1, wherein to normalize the plurality of historical chemical test results comprises to perform absolute normalization of the historical test results based on a predetermined threshold value, to perform conditional normalization of the historical test results for a predetermined test condition, and/or to average the absolute normalization and the conditional normalization.

6. The computing device of claim 1, wherein to train the chemical composition predictor comprises to categorize each chemical component of each formulation of the normalized test results into a component category based on relative proportion of each chemical component.

7. The computing device of claim 1, wherein to train the chemical composition predictor comprises to train a first machine learning model to predict a corresponding first component category based on the normalized test results and to train a second machine learning model to predict a corresponding second component category based on the normalized test results and an output from the first machine learning model.

8. The computing device of claim 1, further comprising a formula optimization module to:
filter the normalized test results based on the predicted composition to generate filtered test results, wherein the filtered test results are based on historical specialty chemical tests that involve the plurality of chemical components of the predicted composition;
train a formulation optimization predictor to predict a normalized performance indicator given a test condition and a formulation based on the filtered test results, wherein the formulation is indicative of a percentage composition for each chemical component;
generate a plurality of candidate chemical formulations based on the predicted composition, wherein each candidate chemical formulation is indicative of a percentage composition for each chemical component of the predicted composition; and
predict a predicted normalized performance indicator for each of the candidate chemical formulations given a requested test condition and a respective candidate chemical formulation with the formulation optimization predictor in response to training of the formulation optimization predictor.

9. The computing device of claim 8, wherein to generate the plurality of candidate chemical formulations comprises to generate a one-hot encoding of a representation of the plurality of candidate chemical formulations.

10. The computing device of claim 8, wherein the formula optimization module is further configured to identify a top performing candidate chemical formulation based on the predicted normalized performance indicator.

11. The computing device of claim 8, wherein the formula optimization module is further to:
receive additional test results associated with the top performing candidate chemical formulation; and
re-train the formulation optimization predictor based on the additional test results.

12. A method for specialty chemical formulation development, the method comprising:
normalizing, by a computing device, a plurality of historical specialty chemical test results to generate normalized test results, wherein each normalized test result is indicative of a test condition and a normalized performance indicator;
training, by the computing device, a chemical composition predictor to predict a plurality of chemical components of a formulation given a test condition and a normalized performance indicator based on the normalized test results; and
predicting, by the computing device, a predicted composition given a specified test condition and a specified normalized performance indicator with the chemical composition predictor in response to training the chemical composition predictor, wherein the predicted composition is indicative of a plurality of chemical components.

13. The method of claim 12, wherein the test condition comprises pressure, temperature, pH, or bicarbonate (HCO₃) concentration.

14. The method of claim 12, wherein the historical specialty chemical test results comprise corrosion inhibitor test results, and wherein the normalized performance indicator is indicative of a measured corrosion rate scaled between a predetermined minimum value and a predetermined maximum value.

15. The method of claim 12, wherein normalizing the plurality of historical chemical test results comprises performing absolute normalization of the historical test results based on a predetermined threshold value, performing conditional normalization of the historical test results for a predetermined test condition, and/or averaging the absolute normalization and the conditional normalization.

16. The method of claim 12, wherein training the chemical composition predictor comprises training a first machine learning model to predict a corresponding first component category based on the normalized test results and training a second machine learning model to predict a corresponding second component category based on the normalized test results and an output from the first machine learning model.

17. The method of claim 12, further comprising:
filtering, by the computing device, the normalized test results based on the predicted composition to generate filtered test results, wherein the filtered test results are based on historical specialty chemical tests that involve the plurality of chemical components of the predicted composition;
training, by the computing device, a formulation optimization predictor to predict a normalized performance indicator given a test condition and a formulation based on the filtered test results, wherein the formulation is indicative of a percentage composition for each chemical component;
generating, by the computing device, a plurality of candidate chemical formulations based on the predicted composition, wherein each candidate chemical formulation is indicative of a percentage composition for each chemical component of the predicted composition; and
predicting, by the computing device, a predicted normalized performance indicator for each of the candidate chemical formulations given a requested test condition and a respective candidate chemical formulation with the formulation optimization predictor in response to training the formulation optimization predictor.

18. A computing device comprising:
a processor, and
a memory having stored therein a plurality of instructions that when executed by the processor cause the computing device to perform the method of claim 12.

19. One or more machine readable storage media comprising a plurality of instructions stored thereon that in response to being executed result in a computing device performing the method of claim 12.

20. A computing device comprising means for performing the method of claim 12.
